(19) United States
(12) Patent Application Publication
(10) Pub. No.: US 2011/0303222 A1
(43) Pub. Date: Dec. 15, 2011

(54) LIQUID EJECTION HEAD AND INHALER

(75) Inventors: Satoshi Tsuchiya, Inagi-shi (JP); Takatoshi Tanaka, Tokyo (JP)

(73) Assignee: CANON KABUSHIKI KAISHA, Tokyo (JP)

(21) Appl. No.: 13/201,322

(22) PCT Filed: Mar. 18, 2010
(86) PCT No.: PCT/JP2010/055300

§ 371 Date: Aug. 12, 2011

(30) Foreign Application Priority Data
Apr. 14, 2009 (JP) 2009-097791

(51) Int. Cl. A61M 15/00; B67D 7/06

(52) U.S. Cl. 128/203.12; 222/478

(57) ABSTRACT

The present invention provides a liquid ejection head that includes a plurality of rows of liquid ejection ports 11 to eject liquid and a plurality of slit-shaped gas jet ports 12 or a plurality of rows of gas jet ports to jet out gas arranged alternately with the liquid ejection ports 11. The group of ejected liquid droplets is prevented from gathering by making the gas flow rate of gas jetted out from the slit-shaped gas jet ports 12 or the rows of gas jet ports arranged at the outermost sides among the plurality of slit-shaped gas jet ports or the plurality of rows of gas jet ports, whichever appropriate, highest.

### TECHNICAL FIELD

The present invention relates to a liquid ejection head to be used for an inhaler designed to eject liquid droplets of a medicine or a pleasure intake item to allow a user to inhale them. The present invention also relates to an inhaler.

### BACKGROUND ART

Inkjet heads of inkjet recording apparatus are required not only to eject liquid droplets but also to control the direction of ejection of liquid droplets. To satisfy this requirement, methods have been proposed to control the direction of liquid droplets by means of one or more air flows.

For example, Japanese Patent Application Laid-Open No. H02-204049 discloses an inkjet head that prevents liquid droplets from deflecting so as to eject liquid droplets along a nearly straight trajectory by arranging air jet ports for driving air to be jetted out in a direction same as the direction of ink ejection at the outside of a row of ejection ports for ejecting ink. Japanese Patent Application Laid-Open No. 2007-301935 discloses an inkjet head showing an improved recording quality by arranging an air jet port substantially laid on an ink ejection port for ejecting ink so as to eject liquid droplets and air together and reduce tails of liquid droplets.

### DISCLOSURE OF THE INVENTION

Liquid ejection heads to be used for inhalers are required to produce an increased number of liquid droplets if compared with liquid ejection heads to be used for inkjet recording apparatus. However, a problem that liquid droplets collide with one another to form large liquid droplets and adhere to the head surface a large extent to clog the ejection port arises when a liquid ejection head designed to merely produce an increased number of liquid droplets is used for an inhaler.

The cause of the above problem is that negative pressure arises in the group of liquid droplets when an increased number of liquid droplets is used. The negative pressure in the group of liquid droplets is high at and near the center of the group of liquid droplets and low at the peripheral part of the group of liquid droplets so that the group of liquid droplets fly to gather at and near the center as illustrated in FIG. 13A. Then, as a result, liquid droplets collide with each other to form large liquid droplets and adhere to the ejection head surface due to distorted trajectories of liquid droplets.

Therefore, an object of the present invention is to provide a liquid ejection head and an inhaler with few liquid droplets colliding with one another or adhering to the surface of the ejection head that can eject a group of micro liquid droplets showing a uniform particle size distribution.

According to the present invention, the above object is achieved by providing a liquid ejection head including: a plurality of rows of liquid ejection ports arranged at intervals; and a plurality of slit-shaped gas jet ports or a plurality of rows of gas jet ports arranged alternately with the rows of liquid ejection ports, in which a gas flow rate of gas jetted out from the slit-shaped gas jet ports or the rows of gas jet ports arranged at the outermost sides among the plurality of slit-shaped gas jet ports or the plurality of rows of gas jet ports, whichever appropriate, is highest.

Thus, according to the present invention, the gas flow rate of gas jetted out from the slit-shaped gas jet ports or the rows of gas jet ports arranged at the outermost sides is made highest to increase the negative pressure of the groups of liquid droplets at the outer sides. Then, as a result, liquid droplets ejected from the liquid ejection ports do not gather toward the center of the group of liquid droplets and hence liquid droplets can be ejected and dispersed as illustrated in FIG. 13B. Thus, when liquid droplets are ejected by a large volume, liquid droplets scarcely collide with one another and adhere to the ejection head surface to enable to eject a group of micro liquid droplets showing a uniform particle size distribution.

Further features of the present invention will become apparent from the following description of exemplary embodiments with reference to the attached drawings.

### BRIEF DESCRIPTION OF THE DRAWINGS

FIG. 1A is a schematic perspective view of an embodiment of liquid ejection head according to the present invention and FIG. 1B is an enlarged schematic perspective partial view, illustrating part of the liquid ejection section of FIG. 1A.

FIG. 2 is a partly broken perspective view of the liquid ejection section of the liquid ejection head illustrated in FIGS. 1A and 1B.

FIG. 3A is a schematic plan view of the liquid ejection head of Example 1, FIG. 3B is a partial perspective view of the liquid ejection head, illustrating the liquid ejection section thereof, and FIG. 3C is an exploded perspective view corresponding to FIG. 3B.

FIG. 4 is a schematic block diagram of the gas supply paths of Example 1.

FIG. 5A is a schematic plan view of a modified liquid ejection head of Example 1, FIG. 5B is a partial perspective view of the modified liquid ejection head, illustrating the liquid ejection section thereof, and FIG. 5C is an exploded perspective view corresponding to FIG. 5B.

FIG. 6 is a schematic block diagram of the gas supply paths of the modified liquid ejection head illustrated in FIGS. 5A through 5C.

FIG. 7A is a schematic plan view of another modified liquid ejection head of Example 1, FIG. 7B is a partial perspective view of the liquid ejection section, and FIG. 7C is an exploded perspective view corresponding to FIG. 7B.

FIG. 8 is a schematic block diagram of the gas supply paths of another modified liquid ejection head illustrated in FIGS. 7A through 7C.

FIG. 9A is a schematic plan view of the liquid ejection head of Example 2, FIG. 9B is a partial perspective view of the liquid ejection section, and FIG. 9C is an exploded perspective view corresponding to FIG. 9B.

FIG. 10 is a schematic block diagram of the gas supply paths of Example 2.

FIGS. 11A, 11B and 11C are schematic plan views of three modified liquid ejection heads of Example 2.

FIG. 12A is a schematic perspective view of the inhaler of Example 3 and FIG. 12B is a schematic elevation thereof in a state where an access cover thereof is opened.

FIG. 13A and FIG. 13B are illustrations of behaviors of groups of liquid droplets ejected from two different liquid ejection heads.

### BEST MODE FOR CARRYING OUT THE INVENTION

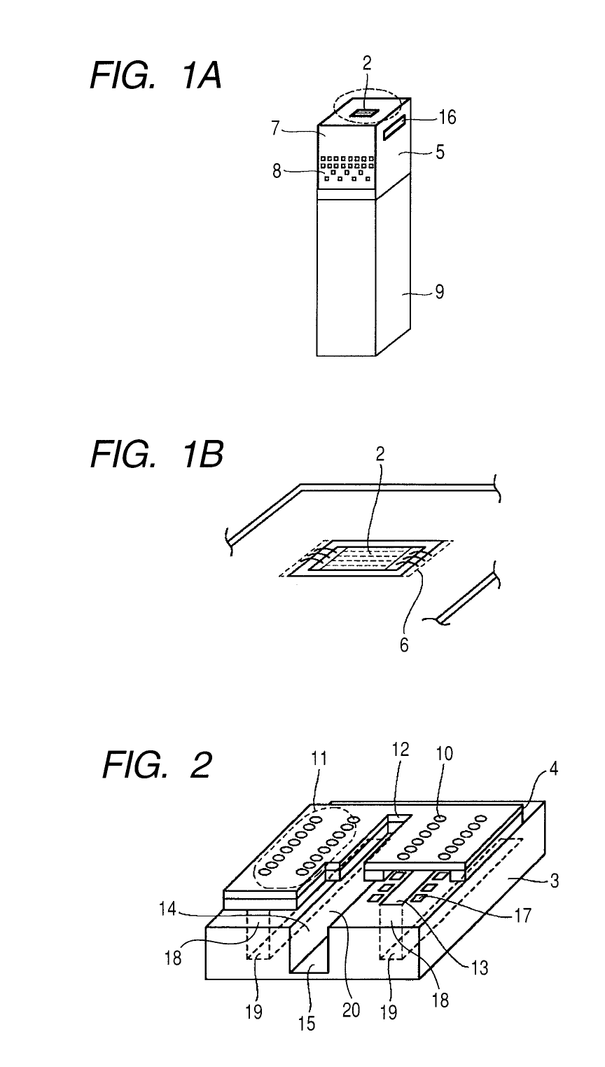

FIGS. 1A and 1B schematically illustrate an embodiment of liquid ejection head, which is of a side shooter type designed to eject liquid droplets in a direction perpendicular to heating elements that generate thermal energy for causing film boiling to take place relative to liquid.

The liquid ejection head of this embodiment includes a liquid ejection section 2, an electric wiring tape 7 and a chip holder 5 and is connected to a liquid tank 9.

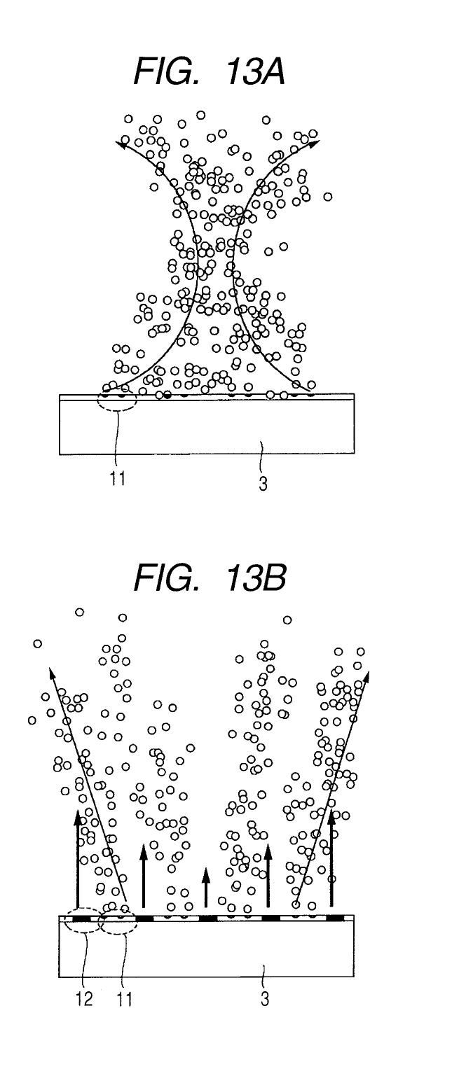

The chip holder 5 is formed by, for example, resin molding. The chip holder 5 has a connection port (not illustrated) for leading liquid from the liquid tank 9 to liquid supply ports 13 (FIGS. 13A and 13B) of the liquid ejection section 2 and also is provided with a function of removably holding the liquid tank 9.

The liquid ejection section 2 is formed so as to include a heat generating element substrate 3 as illustrated in FIG. 2. Liquid supply ports 13 and gas supply ports 14 are long slot type through holes formed by anisotropic etching or laser processing utilizing the crystal orientation of Si in the heat generating element substrate 3 that is a 0.5 to 1 mm thick Si substrate. Each liquid supply port 13 is sandwiched by two rows of heat generating elements 17. The heat generating elements 17 and the electric wiring of Au (not illustrated) for supplying electric power to the heat generating elements 17 are formed by means of a film forming technique. Furthermore, electrode sections for supplying electric power to the electric wiring are arranged at the opposite ends of the rows of the heat generating elements 17. The electric wiring is to apply electric signals for ejecting liquid to a heat generating element substrate 3. The electric wiring connects the electrode terminals 6 that correspond to the electrode sections of the heat generating element substrate 3 and external signal input terminals 8 for receiving electric signals from a main-body.

Path walls for forming liquid paths 18 that correspond to the heat generating elements 17 and gas paths 20 are formed in the heat generating element substrate 3 by means of a resin material and a photolithography technique.

Liquid supplied through the liquid supply port 13 is ejected by the air bubbles generated from the heat generating element 17 from the liquid ejection port 10 provided opposite to the heat generating element 17.

Gas is supplied into a chip holder 5 from a connection port 16 by a pump (not illustrated) and then introduced into the inside from gas introduction ports 15. Then, gas passes through gas paths 20 and jetted out from slit-shaped gas jet ports 12. Note, however, that the gas jet ports 12 may not necessarily be slit-shaped and may alternatively be arranged in rows, each having a plurality of gas jet ports arranged at intervals. Then, each row of gas jet ports may not necessarily draw a straight line and they may alternatively be arranged zigzag so long as they form a row as a whole.

In the liquid ejection head illustrated in FIG. 2, heat generating elements 17 that are energy generating elements for ejecting liquid are arranged on a heat generating element substrate 3. Heat generating elements 17, liquid paths 18, liquid ejection ports 10, slit-shaped gas jet ports 12 (to be referred to as “gas jet ports” hereinafter) form a set as illustrated in FIG. 2 and a plurality of such sets are arranged on a single heat generating element substrate 3. Note that, for the purpose of the present invention, energy generating elements are not limited to the heat generating elements 17 that operate like heaters and oscillation energy generating elements such as piezoelectric elements may alternatively be employed.

A liquid path 18 is surrounded by an ejection port plate 4 where liquid ejection ports 10 for ejecting liquid droplets are formed, a heat generating element substrate 3 and flow path walls defining the gap between the ejection port plate 4 and the heat generating element substrate 3.

A gas path 20 is formed by an ejection port plate 4 where a gas jet port 12 for jetting out gas is formed, a heat generating element substrate 3 and flow path walls defining the gap between the ejection port plate 4 and the heat generating element substrate 3.

According to the present invention, the gas flow rate of gas jetted out from the gas jet ports 12 arranged at the outermost sides of the liquid ejection section 2 of the liquid ejection head 1 is made highest.

### Example 1

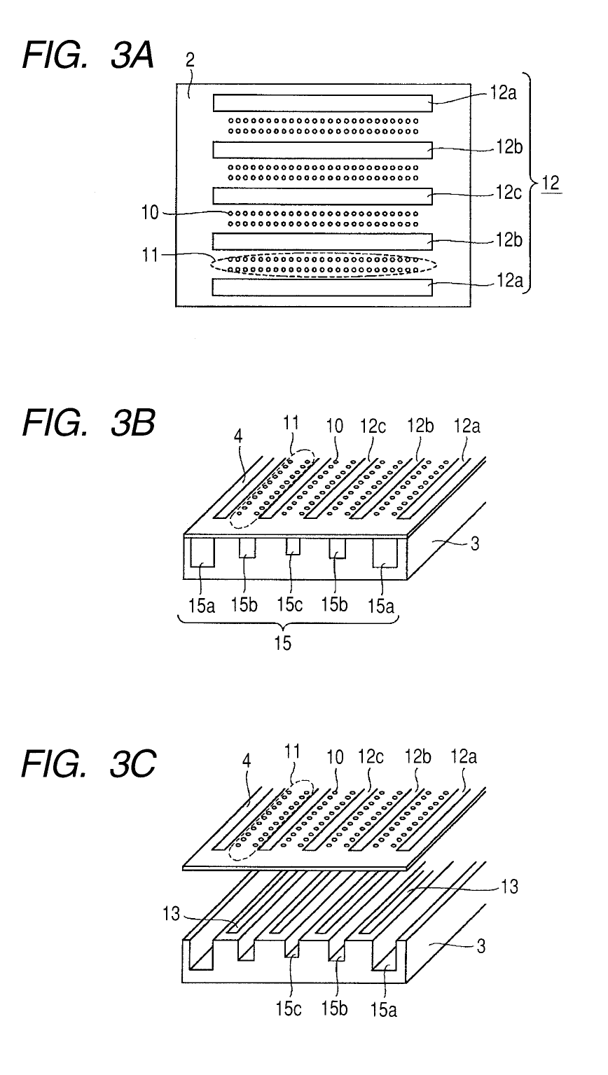

FIGS. 3A through 3C schematically illustrate the liquid ejection head of Example 1. Four rows of liquid ejection ports 11 are arranged in the liquid ejection section 2 and each of the rows has a plurality of liquid ejection ports 10 arranged at intervals. Five gas jet ports 12 (12a, 12b, 12c, 12b, 12a) are arranged alternately with the rows of liquid ejection ports 11. The gas jet ports 12 are arranged in parallel with the rows of liquid ejection ports 11. More specifically, the gas jet ports 12a, 12b, 12c are arranged in the mentioned order from the outside toward the inside of the liquid ejection section 2. The longitudinal dimension of the gas jet ports 12 is preferably greater than the longitudinal dimension of the rows of liquid ejection ports 11 in order to make the gas jetted out from the gas jet ports 12 effectively act on the entire group of liquid droplets. Note that each row of liquid ejection ports 11 may not necessarily draw a straight line and they may alternatively be arranged zigzag so long as they form a row as a whole.

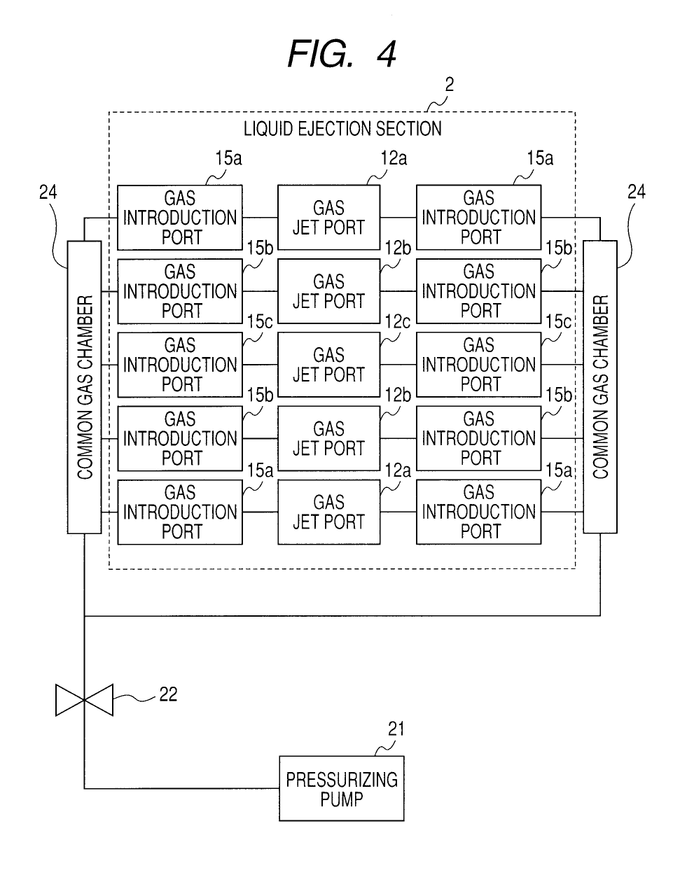

Gas introduction ports 15a, 15b, 15c, 15b, 15a are arranged at the outsides of the opposite ends in the longitudinal direction of each of the rows of gas jet ports 12a, 12b, 12c, 12b, 12a. As illustrated in FIG. 4, gas supplied by means of a pressuring pump 21 can be jetted out from the five gas jet ports 12a, 12b, 12c, 12b, 12a respectively by way of five pairs of gas introduction ports, or ten gas introduction ports 15a, 15b, 15c, 15b, 15a, as illustrated in FIG. 4. All the gas introduction ports 15 of the liquid ejection section 2 are connected to a common gas chamber 24 that is external relative to the liquid ejection section 2 and gas is supplied thereto under the same pressure by means of a single pressurizing pump. A valve 22 is arranged between the pressurizing pump 21 and the gas introduction ports 15a, 15b, 15c, 15b, 15a to control the gas pressure.

The areas of the gas introduction ports 15 are such that the outermost gas introduction ports 15a have the largest area in the liquid ejection section 2 and the inner gas introduction ports 15b, 15c have a smaller area in the liquid ejection section 2. As for the areas of the gas jet ports 12, all the gas jet ports 12a, 12b, 12c have the same area regardless of their positions in the liquid ejection section 2. Thus, when all the gas introduction ports 15 are pressurized under the same pressure by means of the pressurizing pump 21, the gas flow rate of gas jetted out from the outermost gas jet ports 12a in the liquid ejection section 2 shows the highest value due to the pressure loss that varies as a function of the dimensional difference of the gas paths. In other words, the gas flow rate of the inner gas jet ports 12b is smaller and that of the innermost gas jet port 12c is smallest. Due to the difference of gas flow rate, the negative pressure can be made large at the outer side in the liquid ejection section 2 and made small at the inner side in the liquid ejection section 2.

Thus, the liquid droplets ejected from the rows of liquid ejection ports do not gather toward the center of the group of liquid droplets and hence liquid droplets can be ejected and dispersed as illustrated in FIG. 13B. Thus, liquid droplets scarcely collide with one another and adhere to the ejection head surface to enable to eject a group of micro liquid droplets showing a uniform particle size distribution.

The timing of starting jetting out gas from the gas jet ports is preferably before ejecting liquid droplets from the group of liquid ejection ports. As liquid droplets are ejected in a state where gas is jetted out in advance, liquid droplets can be prevented from colliding with one another and adhering to the ejection head surface immediately after the start of ejection.

The gas flow rate of gas jetted out from the outermost gas jet ports 12a in the liquid ejection section 2 shows the highest value and the gas flow rate of the inner gas jet ports 12b and that of the innermost gas jet port 12c are reduced stepwise in the mentioned order in this example. However, the present invention is by no means limited thereto and the effect of suppressing gathering of ejected liquid droplets can be achieved simply by making the gas flow rate of gas jetted out from the outermost gas jet ports 12a highest regardless of the high/low relationship of the gas flow rates jetted out from the inner gas jet ports 12b, 12c.

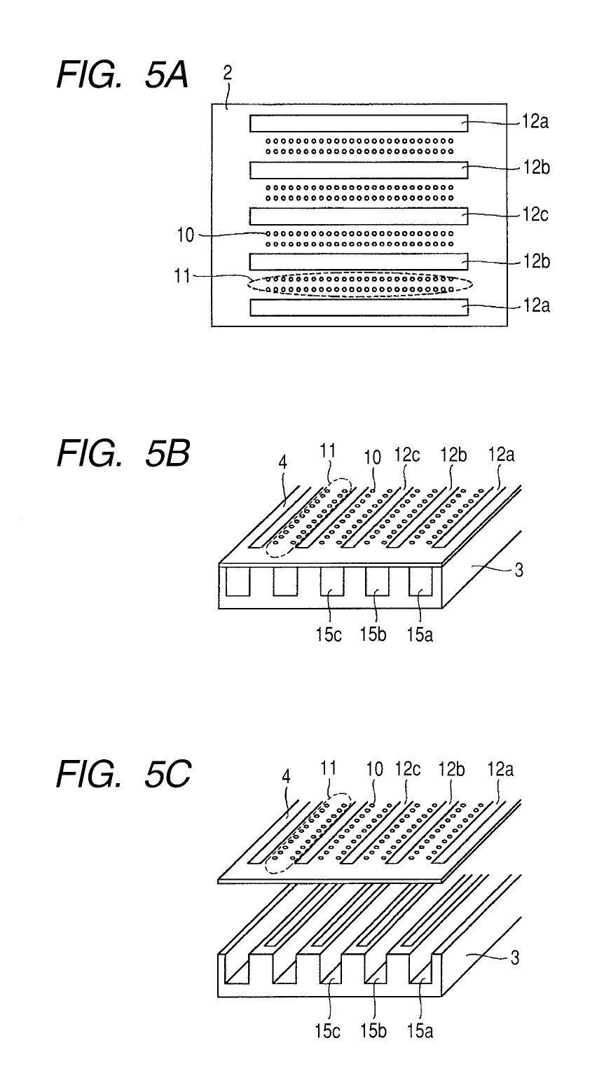

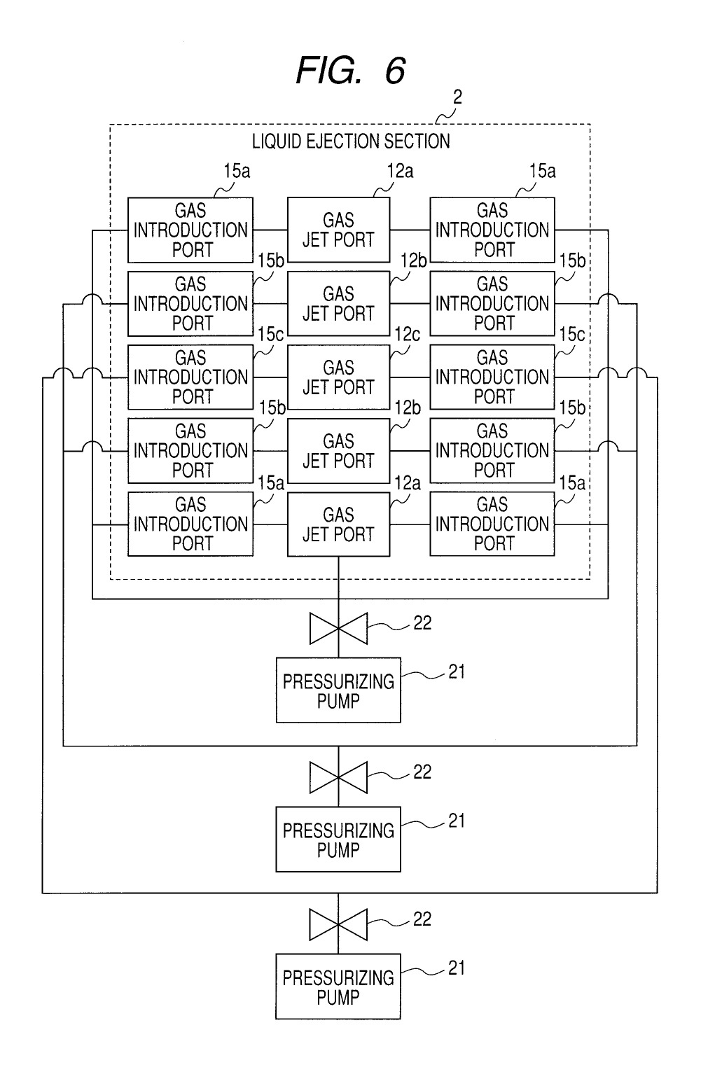

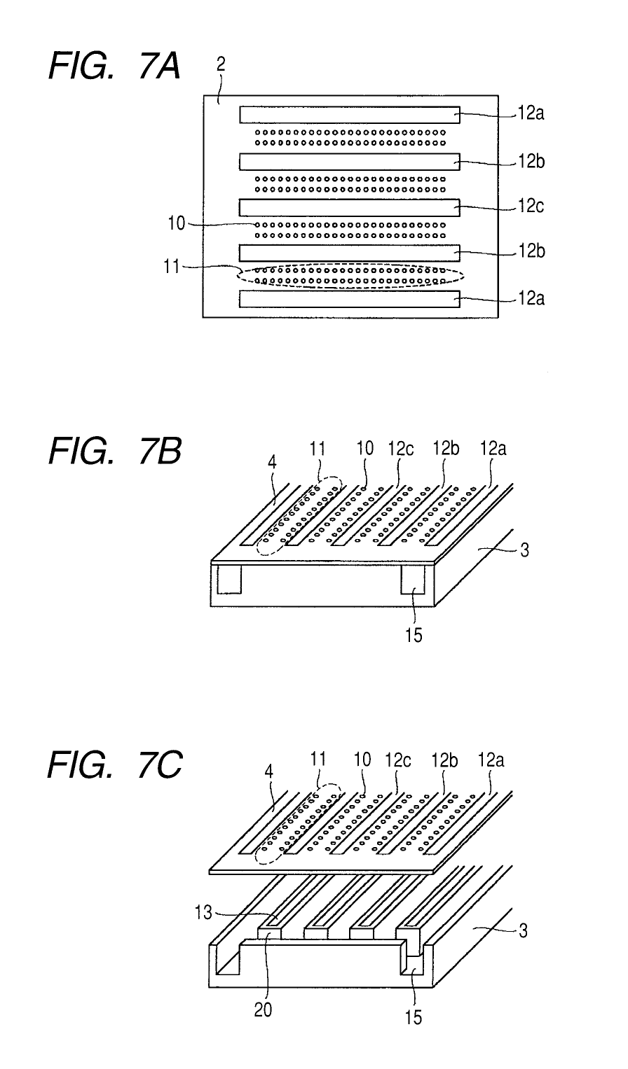

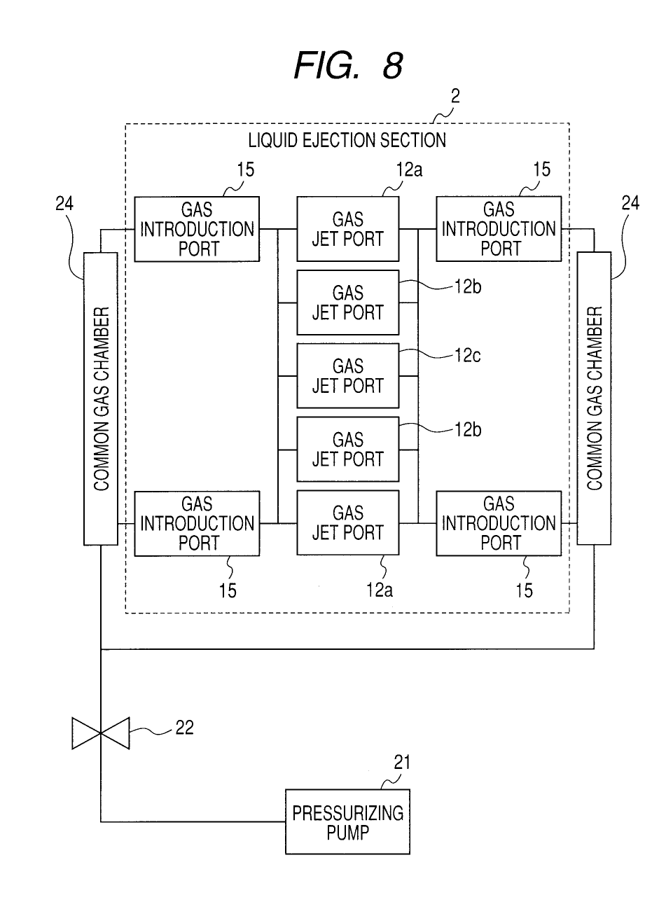

Additionally, the gas flow rates of gas jetted out from the gas jet ports are regulated by way of the areas of the gas introduction ports 15 in this example. However, the present invention is by no means limited thereto and, alternatively, a plurality of pressurizing pumps 21 may be arranged independently at the respective gas introduction ports 15 and the pressures applied to the respective gas introduction ports 15 may be differentiated to regulate the gas flow rates of jetted out gas as illustrated in FIGS. 5A through 5C and 6. Still alternatively, the gas flow rates of jetted out gas may be regulated by utilizing the pressure losses at the gas paths and the gas jet ports 12 as illustrated in FIGS. 7A through 7C and 8.

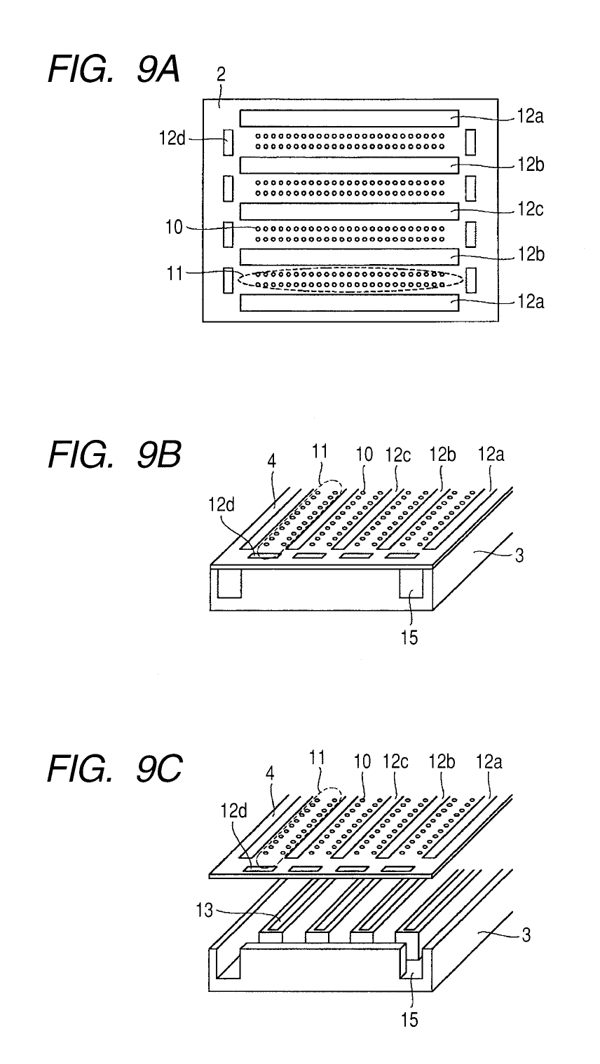

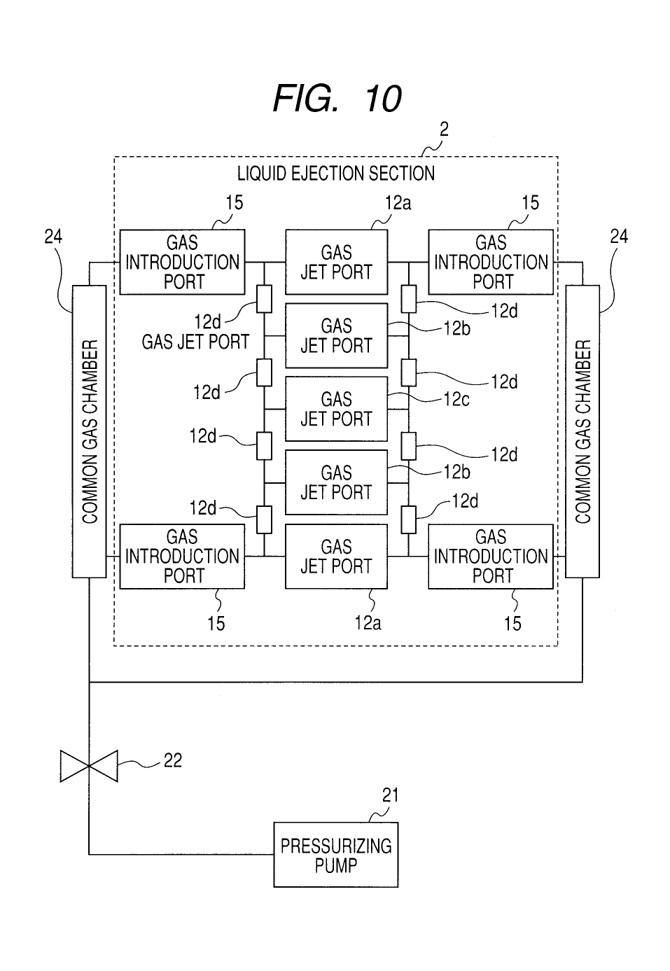

Still alternatively, groups of gas jet ports 12d may be arranged at the opposite ends of the rows of liquid ejection ports 11 that are arranged in parallel as illustrated in FIGS. 9A through 9C and 10. With such an arrangement, liquid droplets ejected from the same row of liquid ejection ports 11 can be prevented from gathering.

### Example 2

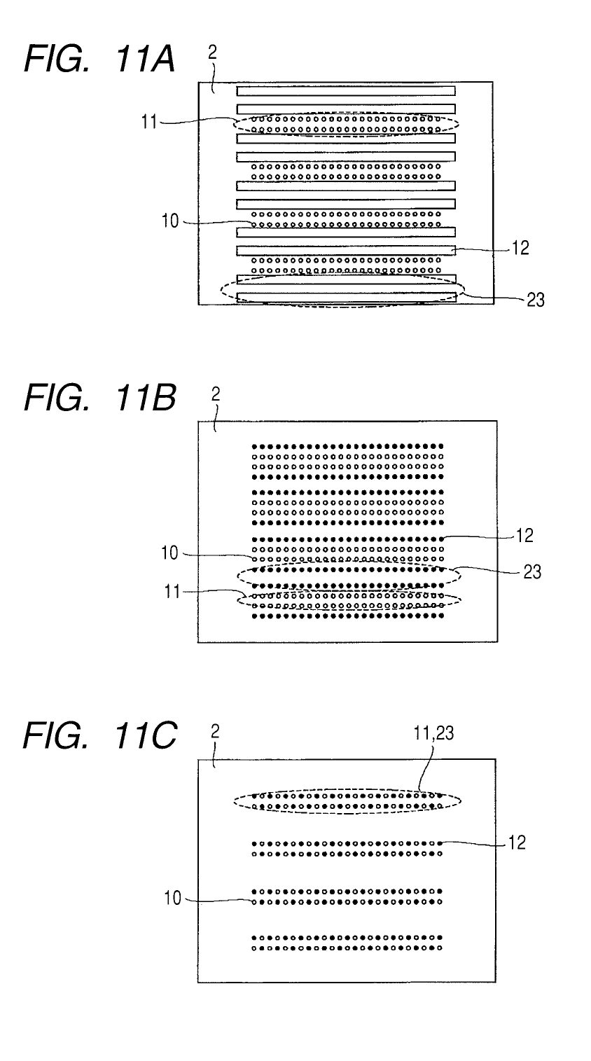

FIG. 11A schematically illustrates the arrangement of the rows of liquid ejection ports 11 and the rows of gas jet ports 23 of the liquid ejection section 2 of the liquid ejection head of Example 2. Four rows of liquid ejection ports 11 are arranged and each of the rows has a plurality of liquid ejection ports 10 arranged at intervals and is sandwiched by rows of gas jet ports 23, each having a plurality of gas jet ports 12, in the liquid ejection section 2. The rows of gas jet ports 23 are arranged in parallel with the rows of liquid ejection ports 11. The longitudinal dimension of the rows of gas jet ports 23 is preferably greater than the longitudinal dimension of the rows of liquid ejection ports 11 in order to make the gas jetted out from the rows of gas jet ports 23 effectively act on the entire group of liquid droplets.

As in the case of Example 1, the negative pressure can be made large at the outer side in the liquid ejection section 2 by making the gas flow rate of gas jetted out from the rows of gas jet ports 23 highest at the outermost sides of the liquid ejection section 2 and made smaller toward the inside of the liquid ejection section 2.

With the above-described arrangement, the liquid droplets ejected from the rows of liquid ejection ports do not gather toward the center of the group of liquid droplets and hence liquid droplets can be ejected and dispersed as illustrated in FIG. 13B. Thus, liquid droplets scarcely collide with one another and adhere to the ejection head surface to enable to eject a group of micro liquid droplets showing a uniform particle size distribution.

The gas jet ports 12 of the rows of gas jet ports 23 may be as small as the liquid ejection ports 10 as illustrated in FIG. 11B. Additionally, rows of gas jet ports 23 can be intermingled with rows of liquid ejection ports 11 by using small gas jet ports 12 as illustrated in FIG. 11C. Then, as a result, liquid droplets ejected from the same row of liquid ejection ports 11 can be prevented from gathering further.

### Example 3

This example relates to an inhaler formed by utilizing a liquid ejection head according to the present invention. The inhaler is linked to a medicine tank and the liquid to be ejected may be selected from protein preparations including insulin, human growth hormone and gonadotropic hormone, nicotine and anesthetics. In the case of inhalation of a medicine such as insulin, the efficiency of absorption by blood is high and appropriate when the particle size of liquid droplets formed from the medicine is about 3 μm is well known. The efficiency of absorption by blood is low and can result in a waste of medicine when the particle size is far from the above appropriate value. Mutual collisions of liquid droplets are reduced to by turn reduce the wasted medicine by utilizing a liquid ejection head according to the present invention.

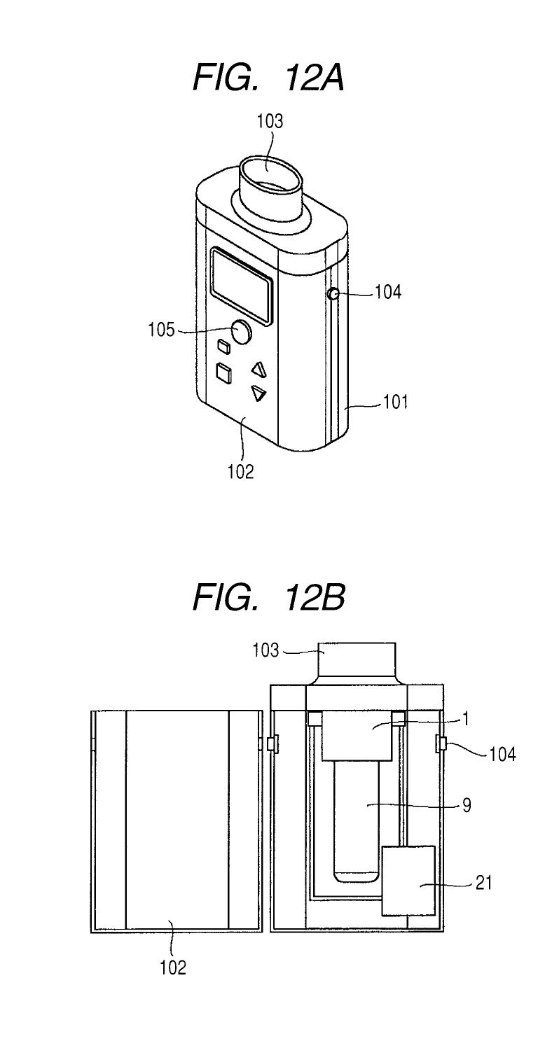

As illustrated in FIGS. 12A and 12B, a main-body case 101 and an access cover 102 form an outer block of a medicine inhaler. The access cover 102 can be opened by unlocking the access cover 102 by means of a lock release button 104. A mount part where a liquid ejection head 1 as illustrated in FIGS. 1A and 1B is mounted is arranged in the inside of the access cover 102. The liquid ejection head 1 can be electrically connected to a drive control section at the mount part of the liquid ejection head 1.

A medicine such as insulin is contained in the liquid tank 9 of the liquid ejection head 1 of the medicine inhaler. The medicine is ejected as liquid droplets from the liquid ejection head 1 into an air flow duct communicating with a mouthpiece 103 so that the user can inhale liquid droplets through the mouthpiece 103.

The flow of operation of the medicine inhaler of this example for medicine inhalation will be described below. The user holds the mouthpiece 103 by lips and depresses an ejection switch 105 while inhaling. External air is introduced into the liquid ejection section 2 from the gas introduction ports 15 by means of a pressurizing pump contained in the medicine inhaler body or by an inhaling action on the part of the user and air starts to be jetted out from the gas jet ports 12 toward the flow paths communicating with the mouthpiece 103. Air is jetted out from the liquid ejection head 1 in a manner as described in detail in Examples 1 and 2. The gas flow rate jetted out from the gas jet ports 12 or the rows of gas jet ports 23 arranged at outer sides of the liquid ejection section 2 is higher. Immediately after air starts to be jetted out, the medicine is ejected as liquid droplets into the liquid paths communicating with the mouthpiece 103 by a drive circuit section. The user inhales from the mouthpiece 103 the liquid droplets ejected into the liquid paths. As a required amount of medicine is ejected, the drive circuit stops operating and transmits a signal for notifying the user of the end of inhalation. The user ends the inhalation, recognizing the signal of the end of inhalation. The flow of operation for inhalation ends within 1 to 2 seconds in any case.

The above-described medicine inhaler of this example can eject a group of micro liquid droplets showing a uniform particle size distribution with few liquid droplets combined together or adhering to the surface of the ejection head so that the user can inhale a medicine with little waste of medicine.

Preferably, the gas jetted out from the gas jet ports 11 are controlled in terms of the content of gas produced when liquid is gasified in the liquid ejection head so that liquid droplets may not be gasified easily. If liquid droplets are water droplets, preferably, the humidity rate of gas is controlled so as not to reduce the particle size of liquid droplets if they are partly gasified. The gas that is jetted out preferably shows a humidity rate of not less than 80% and more preferably a humidity rate of not less than 90%.

### INDUSTRIAL APPLICABILITY

A liquid ejection head according to the present invention can find applications in visualizing devices for visualizing the state of a flowing field or existence of chemicals by using a mist of liquid droplets and image projection apparatus using a mist of liquid droplets as screen in addition to heads for medicine inhalation.

The present invention is not limited to the above embodiments and various changes and modifications can be made within the sprit and scope of the present invention. Therefore, to apprise the public of the scope of the present invention, the following claims are made.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-097791, filed Apr. 14, 2009, which is hereby incorporated by reference herein in its entirety.

1. A liquid ejection head comprising:
a plurality of rows of liquid ejection ports arranged at intervals; and
a plurality of slit-shaped gas jet ports or a plurality of rows of gas jet ports arranged alternately with the rows of liquid ejection ports,
wherein a gas flow rate of gas jetted out from the slit-shaped gas jet ports or the rows of gas jet ports arranged at the outermost sides among the plurality of slit-shaped gas jet ports or the plurality of rows of gas jet ports is highest.

2. The liquid ejection head according to claim 1, wherein the gas flow rate of jetted out gas is gradually reduced from the outermost gas jet ports or the outermost rows of gas jet ports toward the inner gas jet ports or the inner rows of gas jet ports.

3. The liquid ejection head according to claim 1, wherein gas starts to be jetted out from the slit-shaped gas jet ports or the rows of gas jet ports first and subsequently liquid droplets start to be ejected from the liquid ejection ports.

4. An inhaler for ejecting liquid and causing a user to inhale the liquid, comprising:
an air flow duct for guiding liquid droplets to be inhaled by the user into a mouthpiece through inhalation; and
a liquid ejection head for ejecting liquid droplets into the air flow duct described in claim 1.
